(19) United States
(12) Patent Application Publication
(10) Pub. No.: US 2006/0229573 A1
(43) Pub. Date: Oct. 12, 2006

(54) Adjustable infusion catheter

(75) Inventor: Andrew N. Lamborne, Golden, CO (US)

Correspondence Address: DORR, CARSON & BIRNEY, P.C.;ONE CHERRY CENTER, 501 SOUTH CHERRY STREET, SUITE 800, DENVER, CO, 80246, US

(73) Assignee: McKinley Medical L.L.L.P.

(21) Appl. No.: 11/392,919

(22) Filed: Mar. 30, 2006

Related U.S. Application Data
(60) Provisional application No. 60/669,840, filed on Apr. 8, 2005.

(51) Int. Cl. A61M 5/00; B29C 69/00; B29C 65/00; B28B 5/00; B29C 63/00

(52) U.S. Cl. 604/263; 264/241

(57) ABSTRACT

An adjustable infusion catheter includes a flexible tube containing one or more axial lumens that allows fluid to flow from the proximal end of the catheter to the distal end. A syringe or infusion pump is the usual pressure source for fluid at the proximal end. A plurality of small-diameter holes are provided in a fenestrated area near the distal end of the tube to disperse fluid throughout a targeted region within the patient's body. The length of the fenestrated area of the catheter body is adjusted by a slidable sheath which can be positioned along the length of the fenestrated area so that its exposed length substantially matches the targeted region. The ends of the slidable sheath include a seal portion to prevent leakage around the ends of the sheath. Heat shrinkable plastic material can be used to form the sheath and the end seals.

### RELATED APPLICATION

The present application is based on, and claims priority to the Applicant's U.S. Provisional Patent Application Ser. No. 60/669,840, entitled “Adjustable Infusion Catheter,” filed on Apr. 8, 2005.

### BACKGROUND OF THE INVENTION

1. Field of the Invention

This invention relates generally to the field of infusion catheters. More specifically, this invention relates to an improved device and method for administering a medication or other therapeutic fluid to a targeted region in a patient's body, such that the fluid is dispersed throughout the targeted region.

2. Background of the Invention

Infusion catheters for delivery of medication to a targeted region in a patient's body are well known in the art. These catheters are typically comprised of a flexible tube containing one or more axial lumens that allow fluid to flow from the proximal end of the catheter to the distal end. A source of fluid under pressure, such as a syringe or infusion pump, is connected to the proximal end of the catheter and provides fluid flow to the distal end of the catheter, which is inserted into the patient's body. The distal portion of the catheter is provided with one or more exit holes that create fluid communication between the fluid-carrying axial lumen(s) and the portion of the patient's body that surrounds the exterior of the catheter. As seen in the prior art, these exit holes may take a wide variety of forms such as an opening at the end of the axial lumen, holes or slits cut through the side wall of the lumen or tube, spaces between the coils of a spring wound to form a tube, or microscopic openings through a porous membrane shaped to form a tube.

Catheters Providing Even Delivery of Fluid Over an Extended Infusion Segment.

For certain medical treatments, it is beneficial to deliver a slow drip of fluid medication or other therapeutic fluid as evenly as possible over an extended area. For example, infusion of pain medication directly into the surgical site is commonly used to provide post-operative pain management. For surgical procedures involving a long incision or a relatively broad region (several square inches or more) of disturbed tissue, clinical studies have demonstrated improved pain relief when pain medication is infused at a slow rate (typically on the order of magnitude of 1-10 cc/hr), dripping along the full length of the incision or across the entire disturbed region. An infusion catheter that only provides a few exit holes is incapable of providing the broad fluid dispersion required in these instances. Simply adding numerous exit holes over an extended length typically results in most of the fluid dripping from only a small number of those holes, thereby depriving adequate fluid contact to other portions of the targeted area and failing to satisfy the clinical need. The prior art shows a variety of infusion catheters that attempt to provide an even dispersion of fluid throughout an extended segment of several inches or more along the length of the catheter. A discussion of several relevant prior art devices follows below.

The Wundcath infusion catheter manufactured by Micor (U.S. Pat. Nos. 6,676,643 and 6,689,110 to Brushey) provides a catheter body comprised of a flexible plastic tube with open proximal end and closed distal end, forming a single axial lumen, with a multitude of holes formed along an extended fenestrated segment near the distal end of the tube. A fine wire coil spring, wound with each adjacent coil touching or nearly touching the next, is positioned within the lumen and extends the full length of the catheter body. The majority of the fluid flowing into the catheter travels down the inside of the wire coil spring, and weeps out between the coils to flow out through the holes in the catheter body. The weeping action caused by the coil spring tends to spread the fluid more evenly between all of the holes along the fenestrated catheter segment, whereas the majority of the fluid would flow out of the first few holes if the coil spring were not in place.

The Soaker catheter sold by I-Flow (U.S. Pat. No. 6,626,885 to Massengale) provides a catheter body comprised of a flexible plastic tube with open proximal end and closed distal end, forming a single axial lumen, with a multitude of holes formed along an extended segment near the distal end of the tube. A microporous tube, made of a porous material formed into a tubular shape with open ends, is positioned within the lumen at the distal end of the catheter and extends slightly further than the fenestrated catheter segment. The majority of the fluid flowing into the catheter travels down the inside of the microporous tube, and weeps out through the micropores to flow out through the holes in the catheter body. The weeping action caused by the microporous tube tends to spread the fluid more evenly between all of the holes along the fenestrated catheter segment, whereas the majority of the fluid would flow out of the first few holes if the microporous tube were not in place.

The UniFlo catheter sold by Sorenson (Merit Medical) (U.S. Pat. Nos. 6,179,816 and 5,957,901 to Mottola et al.) provides a catheter body comprised of a flexible plastic tube with open proximal end and closed distal end, forming a single axial lumen, with a multitude of holes formed along an extended segment near the distal end of the tube. Unlike the Wundcath and Soaker, which use a separate element inside the catheter body to help disperse fluid evenly, the UniFlo controls fluid dispersion along the fenestrated catheter segment by controlling the size of the holes. For a comparably-sized catheter (e.g., 20G diameter with approximately 30 holes over a 5 inch-long segment), the holes in the UniFlo catheter are an order of magnitude smaller than the holes in the Wundcath and Soaker catheters (on the order of 0.001 in. vs. 0.01 in.). The small size of each individual hole, which increases the flow resistance through each hole and thereby reduces the maximum rate of flow through each hole, forces fluid to flow more evenly between all of the holes along the fenestrated catheter segment, whereas the majority of the fluid would flow out of the first few holes if the holes were larger.

A number of other prior art references disclose other catheter configurations that attempt to provide reasonably even dispersion of fluid flow along an extended infusion segment. While most of these prior art devices do not perform as well as the above referenced devices (at least when delivering fluid at relatively slow flow rates) or are significantly more expensive to manufacture, they are hereby incorporated as further examples of means to achieve even fluid dispersion along an extended infusion segment in a catheter.

Catheters Providing an Adjustable-Length Infusion Segment.

For certain medical treatments where fluid medication or other therapeutic fluid is to be delivered over an extended area using an infusion catheter with an extended infusion segment, it would be desirable to be able to match the length of the extended infusion segment to the need at hand. For example, when infusing pain medication along the length of an incision to provide post-operative pain relief, it would be desirable to adjust the length of the fenestrated catheter segment to match the length of the incision, so that medication is delivered along the full length of the incision. The Wundcath, Soaker, and UniFlo catheters described above do not provide any mechanism for adjusting the length, but instead are available in two or three models, each with a different, fixed, fenestrated catheter segment length. Models typically available provide a fenestrated catheter segment of 2.5, 5, or 10 inches in length. The prior art shows a variety of other catheters, typically designed for thrombolysis or infusion of medication to a confined segment inside a blood vessel, that do provide for adjustment of the length of the infusion segment. A discussion of several relevant prior art devices follows below.

The IV catheter taught by Huss et al. (U.S. Pat. No. 4,968,306) is designed for intravenous infusion of medication to a selected segment of a blood vessel at a flow rate of approximately 80 cc/hr. The Huss device provides a catheter having a guide wire; a catheter body formed by an inner and outer elongated tube sealed together at the distal end, such that the guide wire fits inside the inner tube and an annular fluid conduit is formed between the inner and outer tubes; a plurality of exit holes in the outer tube that create fluid communication between the fluid-carrying annular conduit and the region outside the catheter body; and a sliding sheath that fits over the catheter body and slides along the length of the catheter body, such that a selectable portion of the fenestrated catheter segment can be covered or uncovered.

For several reasons, the Huss device is not practical for certain medical applications such as delivery of anesthetic agents to a surgical site for post-operative pain management. First, the Huss device does not provide a means for ensuring even distribution of fluid along the fenestrated catheter segment. This is not an issue at high flow rates in the 80 cc/hr range (the intended use of the Huss device), but is an issue at the low flow rates in the 1-10 cc/hr range typically used for delivering anesthetic agents for post-operative pain relief.

Second, the Huss device does not provide a means for adequately sealing the sliding sheath against the catheter body. The device is described as typically having a sheath ID of 0.059 in. and a catheter body OD of 0.059 in. Such a “line-to-line” fit may provide an adequate seal for short bursts of fluid infusion in the 80 cc/hr range (the intended use of the Huss device), but will not provide an adequate seal for slow infusions that continue for hours or days. When normal manufacturing tolerances are taken into account, gaps of at least 0.001 in. and more likely up to 0.005 in. or more would be expected, providing enough of a leak path for the covered exit ports to provide a substantial amount of fluid flow, which will drip out the end of the sheath. Providing an interference or compression fit between the sheath and the catheter body is necessary to ensure a good seal, but is impractical in the Huss design because the parts could not be assembled if sized with an interference fit.

The Huss device is relatively expensive to manufacture, due to the large number of components, the tolerances required on the components, and the processes used to assemble the components. The manufacturing cost of the Huss catheter may be acceptable for its intended use in treating life-threatening vascular thrombosis, where a catheter selling for hundreds of dollars or more is accepted in the marketplace, but it is not acceptable for applications such as delivery of anesthetic agents for post-operative pain management, where the device must be produced in the $1-10 range to be cost competitive.

SociDal Finally, the Huss device includes a tightenable collar at the proximal end of the sheath. This collar is twisted to tighten down on the catheter body to seal the proximal end against leakage (note the need for this feature is further evidence that the design of the sheath itself does not provide for a good seal against the catheter body). In addition to being an added expense, the design of this collar creates a bulky component that reduces patient comfort and convenience. In the post-operative pain management application, the catheter is secured against the patient's skin and left in place for a period of hours or days, during which time the patient is often mobile. Securing the collar against the skin could cause abrasion and irritation to the skin, especially if the patient is moving around and the collar rubs against the skin. The bulk of the collar can also be inconvenient, as any significant protrusion above the skin surface can tend to catch on clothing, dressings, bed linens, etc.

The catheter disclosed by Zhan et al. (U.S. Pat. No. 5,626,564) is similar to the Huss device and suffers the same shortfalls when evaluated against the present invention. The device disclosed by Ouriel et al. (U.S. Pat. No. 6,755,813) provides yet another similar device also suffering some of the same shortfalls.

The catheter taught by Elsberry (U.S. Pat. Nos. 6,594,880, 6,093,180 and 6,056,725) is designed for infusion of medication to a parenchymal target, such as in treatment of a brain tumor, Alzheimer's disease, or other neurological applications. This catheter design is typically implanted in the patient's body for long-term treatment using an implanted infusion pump. The Elsberry device provides a catheter having a closed-end porous tube held in the open end of a second, non-porous tube. The second tube is formed of a material that expands when heated or exposed to a specific chemical, then returns to its original shape when the heat or chemical is removed. When the second tube is expanded, the user can slide the porous tube in or out to match the exposed length to the size of the parenchymal target; the heat or chemical is then removed and the second tube tightens over the first tube to hold it in the adjusted position. For several reasons, the Elsberry device is not practical for certain medical applications such as delivery of anesthetic agents to a surgical site for post-operative pain management.

The Elsberry device requires that the user apply a controlled amount of heat or a chemical solvent prior to adjusting the length of the infusion segment, then maintain the adjustment position and wait until the expansion effects of the heat or chemical dissipate. This is impractical in a typical surgical setting because: (a) a controlled heat source or specific chemical solvent is not normally available in the operating room, and would thus have to be specially provided at added cost and inconvenience, and (b) clinician and operating room time are typically at a premium, with high associated cost, therefore the added time needed to perform the adjustment steps is not cost effective.

In addition, the Elsberry device teaches a “zero tolerance” (i.e., “line-to-line”) fit between the porous tube and the second tube, and the porous tube does not extend to the proximal end of the catheter (where it could be directly affixed to the catheter connector) but rather is held in place only by contact with the second tube. This may provide adequate fixation for the delicate positioning and manipulation involved with implanting a catheter in the brain, and implantation of the catheter may eliminate the majority of the external forces that could tend to dislodge the catheter from its placement. However, in applications such as delivery of anesthetic agents to a surgical site for post-operative pain management, the catheter is exposed to significant external forces during placement and removal, and also during use (especially if the patient is mobile). A catheter of the Elsberry design, if used in these types of applications, would likely suffer inadvertent separation of the porous tube from the rest of the catheter either during use or during removal, requiring follow-up surgery to remove the portion left inside the patient's body.

The Elsberry device is limited in the choice of materials for the second tube to those that will expand significantly when exposed to heat or a specific chemical, then return to the original shape when the heat or chemical is removed. Elsberry teaches the potential material options as polyacrylonitrile, silicone elastomer, or polyurethane. Catheters used for applications such as delivery of anesthetic agents to a surgical site for post-operative pain management typically require a combination of high tensile strength, high elongation, kink resistance, flexibility and lubricity. In the small sizes typically used for these types of applications (19-21G catheters being most commonly used), silicone and polyacrylonitrile will not provide an adequate combination of these properties. Some polyurethanes are useful for catheters for these applications, but it is unlikely that the material could be optimized for both the material properties needed for these applications and the chemically-induced expansion properties needed for adjustability.

There exists an unmet need for an infusion catheter that delivers fluid along an extended-length infusion segment, provides even dispersion of the fluid delivery along the full length of the infusion segment, and allows the user to easily adjust the length of the infusion segment at the time of use. To provide broad applicability for use in a wide range of surgical procedures, this improved infusion catheter must function well when provided with a suitably long infusion segment of at least 10-12 inches and a suitably small catheter diameter of approximately 19-21G, and when used with an infusion system that delivers fluid at a relatively slow flow rate in the 1-10 cc/hr range. Further, the manufacturing cost for this improved catheter must not be significantly higher than the cost for the referenced Wundcath, Soaker and UniFlo prior art catheters.

### SUMMARY OF THE INVENTION

The present invention provides an infusion catheter and method of use thereof that disperses fluid throughout a targeted region by providing exit holes along an extended section of the distal portion of the catheter. The extended section can be adjusted by the user so that the fluid-dispersing section can be adjusted from a relatively short length to a relatively long length as dictated by the requirements of the application at hand. This provides an adjustment mechanism that is inexpensive to manufacture, easy to use, comfortable and convenient for the patient, and provides even dispersion of the fluid infusion along the fluid-dispersing catheter segment at low flow rates and low fluid-driving pressures.

The present catheter provides an elongated, flexible, tubular catheter body with an axial lumen extending from the proximal end to the distal end. A distal portion of the catheter body is fenestrated with fluid passageways extending from the lumen through the catheter body walls, providing a multitude of pathways for expulsion of fluid from inside the catheter body to the area outside the fluid body. An exterior, sliding sheath is formed of a flexible tube with inside diameter equal to or slightly larger than the outside diameter of the catheter body. The ends of the sheath are necked down to an inside diameter slightly smaller than the outside diameter of the catheter body, so that when the sheath is fitted over the catheter body, the necked down sheath ends form a fluid-tight but slidable seal against the outside of the catheter body. The length of the sheath is greater than the length of the fenestrated section of the catheter body, but shorter than the portion of the catheter body proximal to the fenestrated section. When the sheath is slid distally to cover the entire fenestrated section, all of the fluid passageways are covered and fluid in the lumen cannot be expelled outside the catheter. When the sheath is slid proximally to uncover a portion or all of the fenestrated section, the fluid passageways are uncovered and fluid can be expelled from the lumen through each uncovered passageway. By adjusting the position of the sheath, the user can selectively uncover the desired portion of the fenestrated section, to provide an infusion length appropriately matched to the body region targeted for the infusion.

In the preferred embodiment, the catheter body is formed of an extruded polymeric tube, with a closed end formed at the distal tip, and the fluid passageways are formed by a series of micro-holes passing through the wall of the tube. A plurality of micro-holes is provided along a predetermined length of the catheter body (the fenestrated section). The size and number of the micro-holes are chosen to ensure even dispersion of fluid throughout the fenestrated section.

In the preferred embodiment, the sheath is formed of an extruded, heat-shrinkable polymeric tube. Short segments at the proximal and distal ends of the sheath are shrunk using selectively-applied heat during the manufacturing process, to provide a fluid-tight seal between the sheath and the catheter body.

In another embodiment, the ends of the sheath can be formed with relatively thick circumferential end rings to form the seal between the sheath and the outer surface of the tubular catheter body. In addition, the lubricity of the sheath material and/or the catheter body can be increased to allow the sheath to better slide along the catheter body and still provide the necessary fluid seal.

In the preferred embodiment, the proximal end of the catheter body connects to a standard connector such as a Tuohy-Borst connector or a Luer lock connector, which mates to the distal connection on the fluid source.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a pictorial view of the adjustable infusion catheter according to the present invention showing the catheter connected to an infusion pump and delivering liquid medication across the length of a surgical wound site;

FIG. 2 is a pictorial view of another embodiment of the adjustable infusion catheter incorporating two legs of the catheter body for simultaneous infusion into two separate infusion sites;

FIG. 3 shows a pictorial view of the adjustment of the sheath and sliding of the sheath along the catheter body with the entire fenestrated section exposed;

FIG. 4 is a pictorial view showing the sheath covering approximately half of the fenestrated section;

FIG. 5 is a pictorial view showing the entire fenestrated section of the catheter covered by the sheath;

FIG. 6 is a pictorial view which depicts the flow of liquid medication out of the catheter when the sheath is positioned to expose the entire fenestrated section;

FIG. 7 is a pictorial view of the adjustable infusion catheter showing the sheath covering a portion of the fenestrated section;

FIG. 8 is a pictorial view showing the entire fenestrated section of the adjustable infusion catheter covered so that there is no flow;

FIG. 9 is an enlarged cross-sectional view of the distal portion of the adjustable catheter having micro-holes forming the fenestrations;

FIG. 10 is an enlarged cross-sectional view of the distal portion of an alternate body of the catheter with a coil positioned in the axial lumen and large holes forming the fenestrations;

FIG. 11 is an enlarged cross-sectional view of another embodiment of an adjustable catheter with a porous tube positioned within the axial lumen and large holes forming the fenestrations;

FIG. 12 is an enlarged cross-sectional view of another embodiment of the adjustable catheter with the distal portion of the catheter body formed from a porous material;

FIG. 13 is an enlarged cross-sectional view of another embodiment of the adjustable catheter showing the tip of the catheter body formed into a bulb in order to prevent the sheath from being displaced from the end of the catheter;

FIG. 14 shows an enlarged cross-sectional view of the proximal end of the fenestration section of the adjustable catheter with a raised diameter segment around the periphery of the catheter body to keep the sheath from being dislodged over the distal end of the catheter;

FIG. 15 is an enlarged cross-sectional view of the body of the adjustable catheter having graduation markings to visually indicate the sheath position along the catheter body and the depth of the catheter placement inside the patient's body;

FIGS. 16-19 are pictorial views showing a method of using the catheter to deliver liquid medication to the targeted region within a patient's body wherein the sheath is positioned to match the fenestrated section of the catheter to the target infusion site with the catheter then inserted through an inducer into the target infusion site; and

FIGS. 20-21 is a pictorial view showing the priming of the catheter with fluid and then the connection of the catheter to an infusion pump for delivery of the liquid medication across the length of the target infusion site.

### DETAILED DESCRIPTION OF THE INVENTION

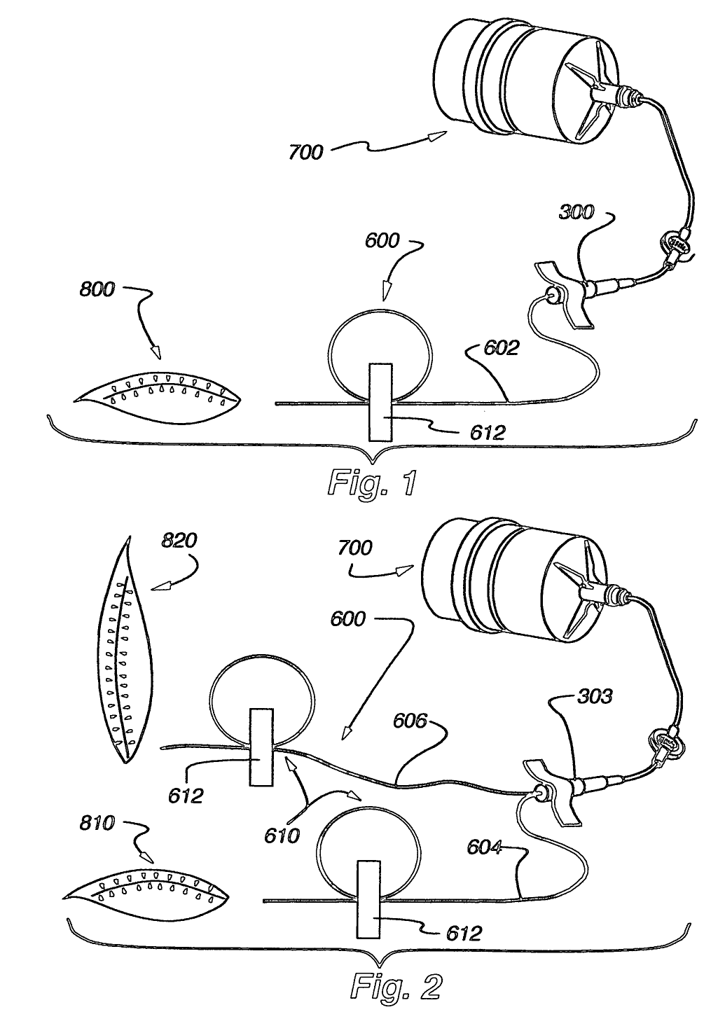

Turning now more specifically to the drawings, FIG. 1 depicts the adjustable infusion catheter of the present invention in use, delivering liquid medication to a surgical wound site 800 in a patient. The adjustable catheter device 600 comprises a flexible, tubular conduit 602 for delivering liquid medication from an infusion device 700 into the target infusion site 800. FIG. 1 shows the infusion device 700 taking the form of the disposable Beeline infusion pump marketed by McKinley Medical LLLP of Wheat Ridge, Colo., but any of a number of devices may be used to provide liquid flow to the catheter including a syringe, gravity-fed infusion bag or bottle, or virtually any of the mechanical or electronic infusion systems commonly used in medical practice. The target infusion site 800 is depicted in FIG. 1 as a surgical incision, but the catheter 600 of the present invention is useful for any targeted infusion site in a patient's body, including specific body structures such as a nerve bundle, an organ, or an area of diseased tissue and body cavities such as an intra-articular space, an abdominal or thoracic space, the interior of a blood vessel, or a surgical site.

FIG. 2 shows an alternate embodiment of the invention, a dual-leg adjustable catheter device 610 incorporating two legs 604, 606 for infusion into two sites, 810 and 820, respectively. Each leg is independently adjustable, allowing the user to match two infusion sites of differing size. As shown in FIG. 2, the lower leg 604 of the catheter is adjusted to match the smaller incision 810 while the upper leg 606 is adjusted to match the longer incision 820.

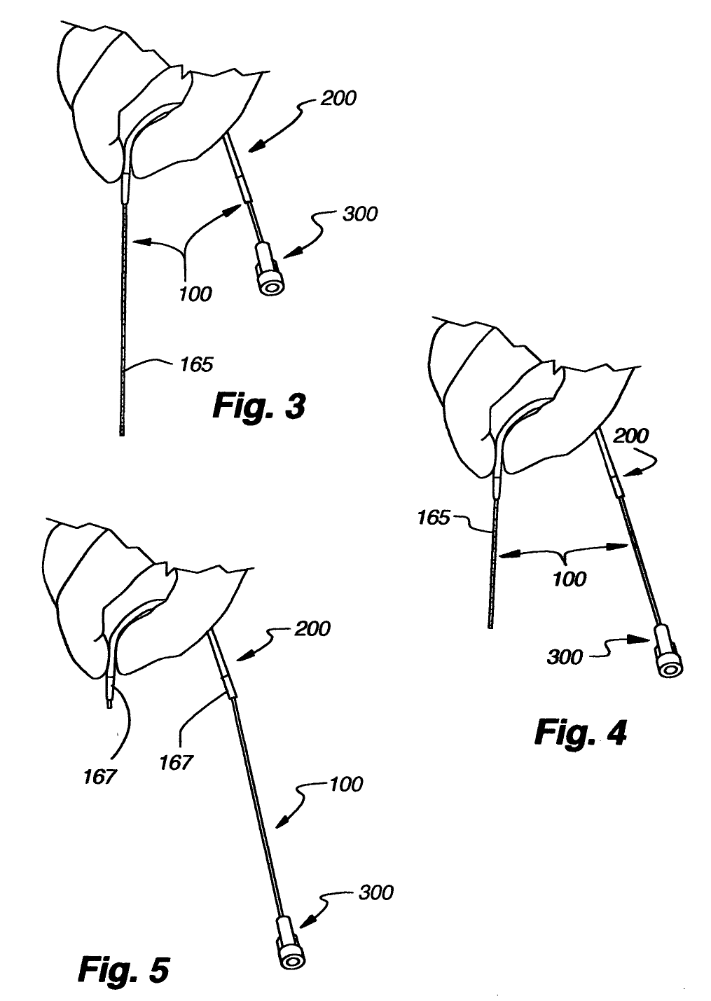

FIGS. 3-5 illustrate adjustment of the infusion catheter. The major components of the adjustable infusion catheter are the catheter body 100, the sheath 200, and the proximal connector 300. The catheter body 100 is a length of flexible tubing having fenestrations 165 along a section of the distal portion of the tubing. The proximal end of the catheter body 100 is connected in fluid-tight fashion to the proximal connector 300, forming a fluid conduit from the proximal connector down through the bore of the catheter body and out through the fenestrations 165. The sheath 200 is a length of flexible tubing formed from a suitable plastic such as a heat-shrinkable polymer. The sheath 200 is sized so that, when in the “expanded” condition prior to heat shrinking, the inside diameter of the sheath fits around the outside diameter of the catheter body 100 with at least some minimal clearance so as to allow the sheath to slide over the catheter body. The sheath 200 is also sized so that, when in the “recovered” condition after heat shrinking, the inside diameter is reduced to a size at least minimally smaller than the outside diameter of the catheter body 100 so as to ensure an interference fit between the sheath and the catheter body that allows for sliding the sheath along the length of the catheter body yet provides a fluid-tight seal 167 between the sheath and the catheter body. The length of the sheath 200 is preferably sized to be at least minimally longer than the length of the fenestrated section of the catheter body, so that the sheath can be adjusted to block the entire fenestrated section, if desired. Alternately, the sheath may be shorter than the fenestrated section, in which case at least a portion of the fenestrated section will always remain uncovered and therefore open to provide fluid flow.

The catheter body 100 is preferably formed of a material that is flexible, suitably biocompatible for prolonged contact with body tissues, cost-effective, and manufacturable with standard catheter production techniques such as extrusion and tip forming. Suitable materials include but are not limited to nylon, polyether block amide, polyurethane, polyimide, PVC, FEP and PTFE.

The sheath 200 is preferably formed of a material that is flexible, heat shrinkable, suitably biocompatible for prolonged contact with body tissues, cost-effective, and manufacturable with standard tubing production techniques such as extrusion. Suitable materials include but are not limited to polyester, PTFE, FEP, and polyolefin.

In the preferred embodiments, the proximal connector 300 is a female luer-lock connector. The proximal connector 300 is preferably formed of a material that is suitably biocompatible for contacting fluid that is then delivered to body tissues, is cost effective, and is manufacturable with standard production techniques such as injection molding and solvent or adhesive bonding. Suitable materials include but are not limited to acrylic, polycarbonate, ABS, PVC, polyethylene and polypropylene. The proximal connector may be permanently attached to the catheter body, such as a female luer-lock connector adhesively bonded to the catheter body, or it may be removably connected to the catheter body, such as a Tuohy-Borst connector.

The user adjusts the position of the sheath 200 along the catheter body 100 by grasping the sheath and pulling it in the desired direction. The catheter body or proximal connector is also held to provide tension when sliding the sheath, but this is omitted from the illustration to provide a better view of the device. As the sheath is slid distally, the fenestrated section 165 of the catheter body is partially or completely covered by the sheath. The degree of coverage is dependent on the axial position of the sheath. In FIG. 3, the device is shown with the sheath 200 positioned along the proximal portion of the catheter body 100, such that the entire fenestrated section of the catheter body is exposed. In FIG. 4, the sheath 200 has been slid distally along the catheter body 100 such that the sheath 200 is covering a portion of the fenestrated section 165 of the catheter body. In this position, the sheath 200 blocks flow from the covered fenestrations 165, so fluid can only flow out of the uncovered portion of the fenestrated section. In FIG. 5, the sheath 200 has been slid further so that the distal end of the sheath is very near the distal end of the catheter body 100 and covering the entire fenestrated section 165 of the catheter body. In this position, the sheath blocks flow entirely because all of the fenestrations are covered.

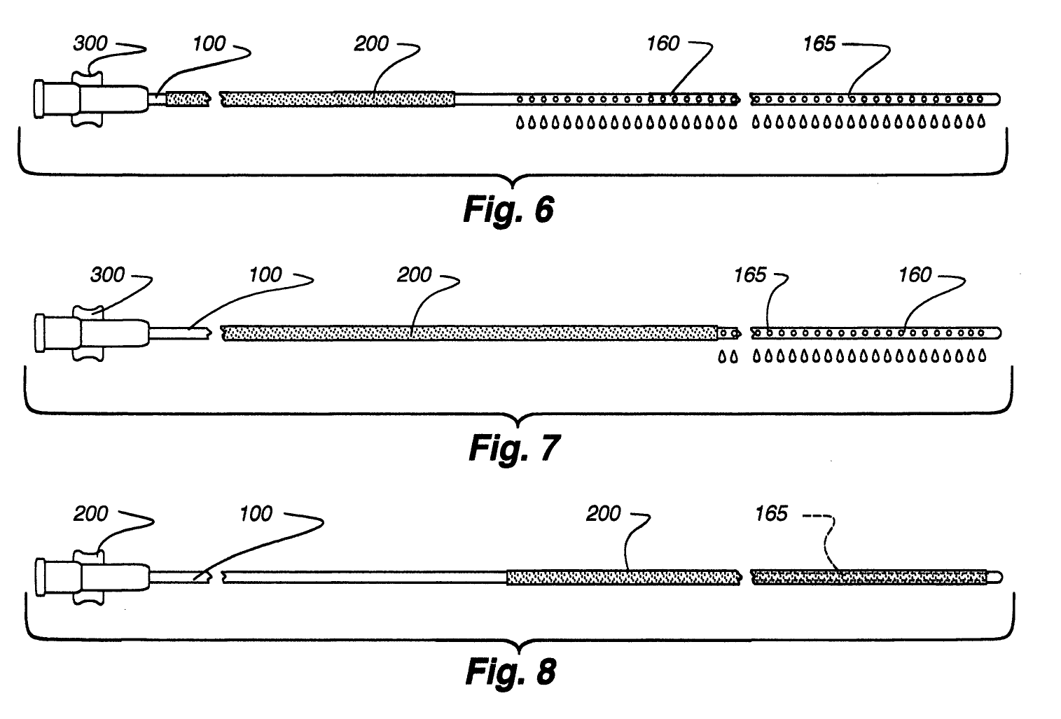

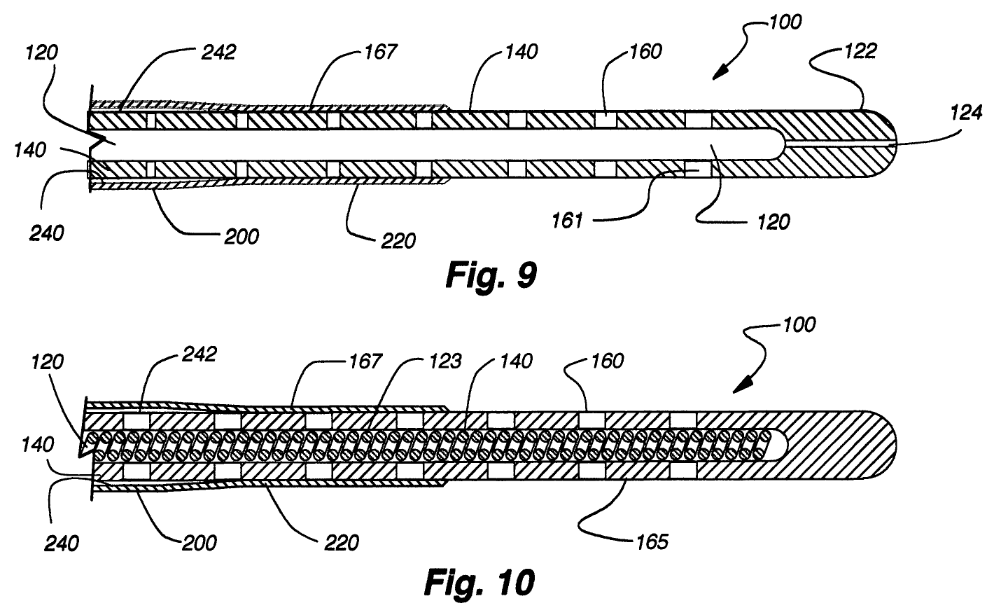

FIGS. 6-8 depict the fenestrated section 165 of the catheter body 100 in greater detail, and show the resulting pattern of fluid flow from the device when the sheath is adjusted to the same positions depicted in FIGS. 3, 4 and 5, respectively. FIG. 6 illustrates the device with the sheath 200 slid to the proximal portion of the catheter body 100, such that the entire fenestrated section 165 of the catheter body is exposed. In this position, fluid delivered to the catheter from the infusion device via the proximal connector 300 drips out of the catheter body along the full fenestrated section 165. FIG. 7 illustrates the device with the sheath 200 slid distally to cover a portion of the fenestrated section 165, while leaving the remaining portion of the fenestrated section uncovered. In this position, fluid delivered to the catheter cannot flow out through the covered fenestrations, which are blocked by the sheath, therefore the fluid only drips out of the catheter body along the uncovered portion of the fenestrated section. FIG. 8 illustrates the device with the sheath 200 slid further distally to cover the entire fenestrated section 165. In this position, flow of fluid from the catheter is completely blocked.

Referring now to FIG. 9, the preferred embodiment of the invention is illustrated in greater detail through a cross-sectional view of the distal portion of the catheter. The catheter body 100 takes the form of a closed-end tube 122 forming an axial lumen 120 inside the outer tubular wall 140. Fenestrations 160 provided through the tubular wall 140 and the axial lumen 120 form a fluid conduit from the proximal connector 300 to the infusion site area outside of the distal portion of the catheter. In the preferred embodiment, the size of individual fenestrations 160 are controlled and very small, such that fluid delivered to the catheter from the infusion device flows out through all of the uncovered fenestrations even when such fluid is provided at relatively low flow rates and low infusion pressures. The rate of fluid flow through any individual fenestration 160 is proportional to the size of the opening and the pressure differential from the inside to the outside. If the individual fenestrations 160 are too large, most or all of the fluid will flow out of the most proximally-located fenestrations. By sizing the fenestrations small enough, a small number of fenestrations will not be able to accommodate all of the fluid flow, therefore fluid will be distributed more evenly between all of the uncovered fenestrations.

The actual size and placement of the fenestrations 160 must be selected to balance the conflicting needs of providing a uniform flow distribution throughout the fenestrated area (which requires the fenestration size to be minimized) and ensuring that the flow restriction created by the fenestrations does not cause a clinically significant reduction in the rate at which the fluid is delivered to the infusion site (which requires that the fenestration size be maximized). In the preferred embodiments, the size of each fenestration 160 is in the range of 0.0002 in. to 0.005 in., with optimal fenestration size dependent on the thickness of the catheter body wall 140, the number of fenestrations provided (including the expected range in number of uncovered fenestrations for typical usage), the range of desired flow rates of fluid through the catheter, and the fluid pressure created by the infusion device 700. The size and spacing of individual fenestrations may vary throughout the fenestrated area or section 165 in order to improve flow uniformity; for example, the distal portions of the fenestration section may have more or larger fenestrations 161 to balance the fluid pressure loss as fluid flows distally or to provide for sufficiently low flow restriction when only a relatively small number of fenestrations are left uncovered at the distal end.

While the illustrated embodiments incorporate a closed, rounded tip 122 at the distal end of the catheter body, alternate tip configurations such as a smooth, open tip or a tip with a small fenestration 124 at the end are also acceptable. For embodiments where the tip is not closed, the sheath cannot be used to completely stop the infusion as the tubular sheath cannot block the distal tip of the catheter body.

Still referring to FIG. 9, and also to FIGS. 10 through 13, which all show the same detail of the sheath, the distal end of the sheath is shown in side cross-sectional views. The sheath 200 takes the form of an open-ended tube created by a tubular wall 240 of heat-shrinkable material. The majority of the sheath 200 is in the “expanded” form, with an annular gap 242 of at least minimal clearance created between the sheath wall 240 and the catheter body wall 140. At the distal end of the sheath, heat is applied during manufacture to shrink the end seal portion 167 of the sheath wall 240 into a necked-down or seal section 220. As discussed above, the “recovered” condition of the sheath tubing after heat-shrinking provides a diameter inside this necked-down section 220 that is smaller than the outside diameter of the catheter body 140. However, because the catheter body is in place under the sheath, the necked-down section 220 cannot reach the fully recovered diameter condition but rather is forced to maintain the slightly-stretched diameter condition wherein the necked-down sheath ID matches the catheter body OD. This condition creates a squeeze seal or interference fit between the inside diameter of the necked-down section 220 and the outside diameter of the catheter body, with residual stress in the heat-shrink material due to the incomplete diameter recovery creating a sealing force around the circumference of the catheter body. This interference fit at the necked-down section forms a fluid-tight but slide-able seal 167, such that the sheath can be slid along the catheter body to any desired position along the catheter while maintaining the fluid-tight seal between the sheath and the catheter body. An equivalent necked-down end is also formed at the proximal end of the sheath (not visible in the enlarged section illustrated in FIGS. 9-13, but visible in FIG. 14), such that both ends of the sheath are sealed in fluid-tight but slide-able configuration about the circumference of the catheter body.

In the preferred embodiments, the interference fit or seal 167 between the necked-down sections 220 and the catheter body 100 is in the range of 0.0005 in. to 0.005 in., with optimal interference dependent on the dimensions (such as wall thickness and overall diameter) of the catheter body and the sheath, the modulus and yield strength of the catheter body and sheath materials, the elasticity of the sheath material after heat-shrinking, the coefficient of friction between the catheter body and the sheath, and the maximum potential fluid pressure created by the infusion device 700.

Referring now to FIG. 10, an alternate embodiment is shown in which the fenestrations 160 are comprised of larger-sized holes. As discussed above, larger fenestrations will allow for most or all of the fluid to flow out of the most proximally-located fenestrations, preventing uniform distribution of fluid flow throughout the uncovered portion of the fenestration area. However, this embodiment provides an alternate way of ensuring uniform flow distribution by inclusion of an internal coil 123 inside the catheter body. The internal coil 123 is formed of wire or other filament wound in closely-spaced coils. The very small space between coils serves to limit the maximum flow from the axial lumen 120 inside the coil out through any short section of the coil to the adjacent fenestration, thereby ensuring uniform flow distribution to each of the uncovered fenestrations. The internal coil 123 may extend inside the full length of the catheter body, or may be of a relatively short length extending only inside the fenestrated area of the catheter body.

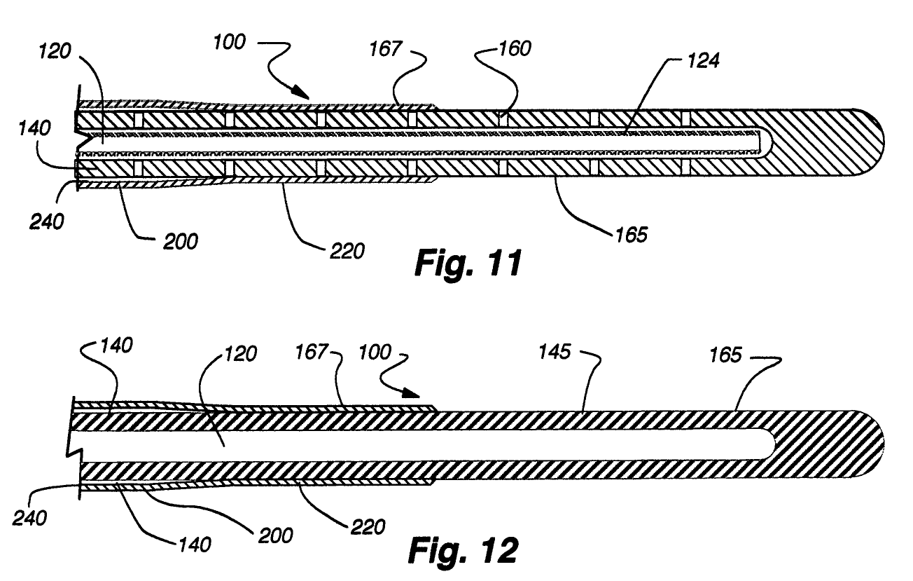

Referring now to FIG. 11, another alternate embodiment is shown in which the fenestrations 160 are comprised of larger-sized holes. In this particular embodiment, an alternate means of ensuring uniform flow distribution is provided by inclusion of an internal porous tube 124 inside the catheter body. The internal porous tube 124 is formed of a micro-porous material such as expanded PTFE or polysulfone with micropore size in the 0.1 to 10 micron range. However, other suitable materials could be readily substituted. The very small size of the micropores serves to limit the maximum flow from the axial lumen 120 inside the porous tube out through any short section of the porous tube to the adjacent fenestration 160, thereby ensuring uniform flow distribution to each of the uncovered fenestrations. The internal porous tube 124 may extend inside the full length of the catheter body, or may be of a relatively short length extending only inside the fenestrated section 165 of the catheter body.

Referring now to FIG. 12, yet another alternate embodiment is shown in which the fenestrated section 165 is comprised of a porous tube segment 145 incorporated into the distal portion of the catheter body 100. The porous tube segment 145 is formed of a micro-porous material, such as expanded PTFE or polysulfone, with micropore size in the 0.1 to 10 micron range. However, other suitable materials could be readily substituted. The very small size of the micropores serves to limit flow from the axial lumen 120 out through any short section of the porous tube, thereby ensuring uniform flow distribution along the uncovered portion of the porous tube segment. The porous tube segment 145 may extend the full length of the catheter body, with a secondary non-porous outer sheath portion 200 of the catheter body wall 140 covering that portion of the porous tube proximal to the fenestrated area 165, such that the secondary non-porous outer wall 200 prevents fluid from flowing out through the portions of the porous tube segment that are proximal to the fenestrated section. Alternately, the porous tube segment 145 may form only the fenestrated section 165, connecting to a non-porous segment of the catheter body wall 140 at the proximal end of the fenestrated area.

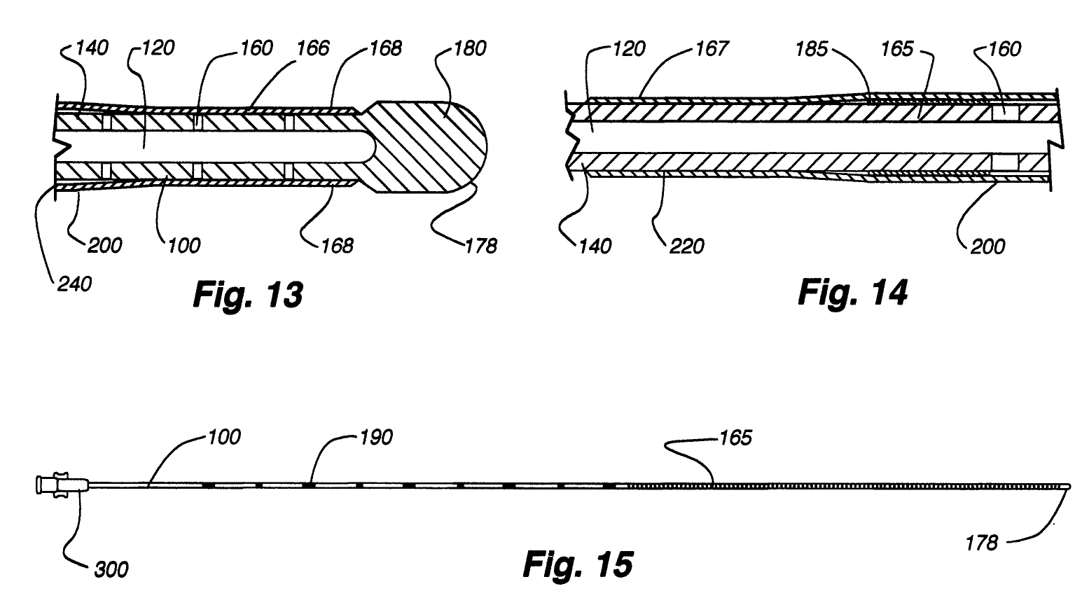

FIG. 13 illustrates a cross-sectional view of the distal portion of the catheter with further detail of the preferred embodiment. The distal tip 178 of the catheter body 100 is shown with a bulbous or raised-diameter feature 180 at the end. The purpose of this feature is to prevent the sheath 200 from sliding off the end of the catheter body 100. This feature improves the user-friendliness of the device, as it can be difficult to get the sheath back over the catheter body once it is slid off, without the special assembly tools that are used during manufacturing.

In addition, the ends 166 of the sheath 200 can have a thickened circumferential end portion 168 to form the fluid seal. The thickened end portion 168 will have an inside diameter that is smaller than the outside diameter of the tube 100. This will still allow the sheath 200 to slide along the surface of the tube 100. The thickened end portion 168 can be used with or without the shrinking of the ends 166 of the sheath seal 200.

FIG. 14 depicts an alternate embodiment of the feature to prevent the sheath from sliding off the distal end of the catheter body. In this configuration, a raised-diameter collar or segment 185 is positioned over the catheter body and under the sheath, near the proximal end of the fenestrated section 165. The necked-down section 220 at the proximal end of the sheath slides up against the raised-diameter segment 185, which acts as a stop to prevent the sheath from sliding further. The raised-diameter segment 185 is positioned to stop the sheath before the distal end of the sheath falls off the distal end of the catheter body. The raised-diameter segment 185 is preferably formed from a short segment of tubing that is bonded in place over the catheter body with adhesive or solvent bonding, or heat-shrinking or other thermal bonding process. Alternately, the raised-diameter segment 185 may be formed directly into the catheter body wall 140, with a process such as RF forming or variable-diameter extrusion.

FIG. 15 illustrates a preferred embodiment of the catheter wherein visual indicator markings or indices 190 are included on the catheter body. The sheath is not shown in FIG. 15, so that the indicator markings 190 can be more clearly seen. The indicator markings 190 are positioned such that the user can determine the position of the sheath (i.e., how long the uncovered portion of the fenestrated section is) and the approximate location of the catheter tip 178 when the distal portion of the catheter is inside the patient and is not visible. The indicator markings 190 are preferably formed directly on the outer surface of the catheter body wall 140 such as by printing with ink or laser marking.

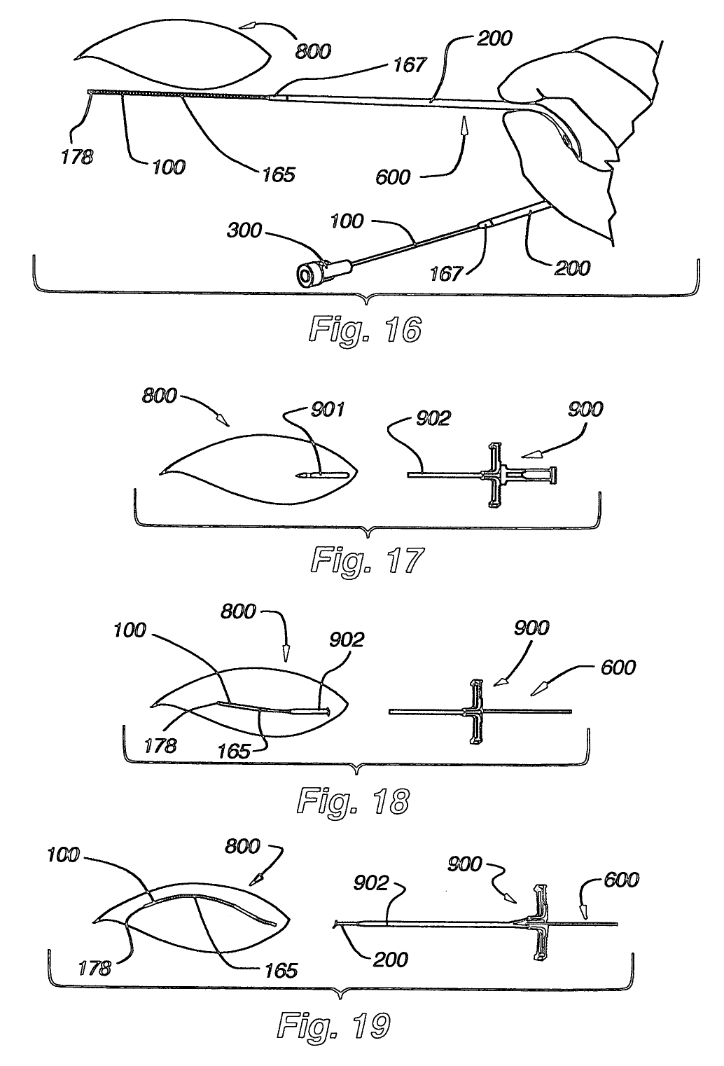

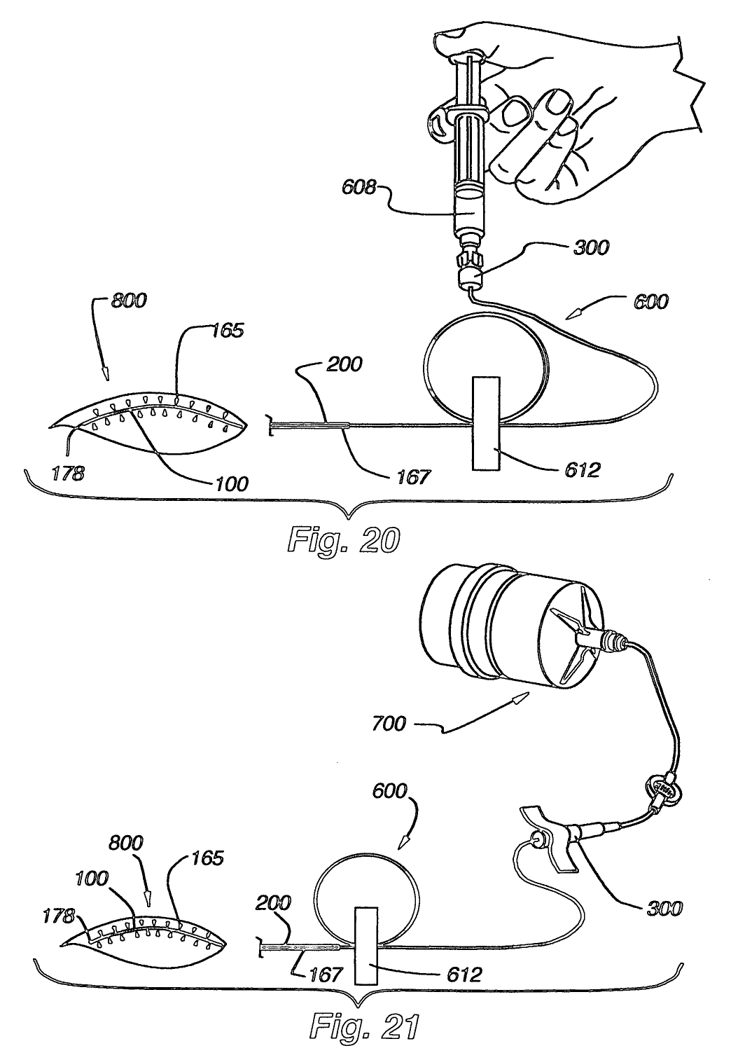

FIGS. 16 through 21 illustrate a method of using the adjustable infusion catheter. FIG. 16 depicts the user sliding the sheath 200 to the desired position along the catheter body 100 such that the length of the exposed portion of the fenestrated section 165 approximately matches the length of the infusion site 800 (depicted as an open incision for illustrative purposes). FIG. 17 illustrates an introducer 900 after the user has inserted it through the patient's skin and into the incision. The introducer 900 is depicted as a peel-away sheath 902 over a sharp needle or stylet 901. FIG. 18 shows the peal-away sheath portion 902 of the introducer still in place in the incision, with the needle/stylet portion 901 removed and the catheter inserted through the sheath and into the incision. FIG. 19 depicts the catheter remaining in place in the incision, as the sheath 902 is withdrawn from the patient and peeled off of the catheter. FIG. 20 illustrates the catheter device 600 in place in the infusion site 800, the catheter body secured to the patient's skin with tape 612, and the user priming the catheter with a fluid-filled syringe 608. Fluid can be seen dripping from the exposed portion of the fenestrated section 165, providing relatively uniform dispersion of fluid throughout the incision. FIG. 21 depicts the entire infusion system in use, with the infusion device 700 connected to the catheter device 600 and fluid being delivered along the length of the infusion site 800.

In the preferred embodiments, both the catheter body and the sheath material are formed of a material with a relatively low coefficient of friction, or are coated with a lubricious coating. This aspect of the invention allows for a heavier interference fit between the necked-down sheath ends and the catheter body, which provides a better seal that remains fluid tight under higher pressures, without requiring an unreasonably high force to slide the sheath along the catheter body. The low-friction material or lubricious coating also reduces the potential for the catheter to stick to bodily tissue or implants inside the patient, thereby reducing the amount of force needed to remove the catheter from the patient's body at the end of the therapy (and associated occurrences of catheter breakage when the user pulls too hard on the catheter).

The sheath is preferably formed of a colored or opaque material 169 that provides high contrast with the color or transparency of the catheter body. This aspect of the invention improves user friendliness by ensuring that the sheath position can be readily determined at a glance.

The catheter of this invention can be made in a wide range of sizes. The preferred size for the catheter is dependent on the clinical application for which it is to be used. The fenestrated section may vary from less than 1 inch long to more than 1 foot long, depending on the body sites that are being targeted. The preferred size for infusion of pain medications into a surgical site, to provide broad applicability for a wide range of surgical procedures, is' a fenestrated section approximately 10-15 inches long with a sheath slightly longer than the fenestrated section. The preferred catheter body size range for infusion of pain medications into a surgical site is between 15G and 24G, with sizes between 18G and 21G most commonly preferred by clinicians. The length of the catheter body must be at least equal the length of the sheath plus the length of the fenestrated section, to provide room for the entire sheath to be positioned proximal to the fenestrated section so all fenestrations are uncovered. The length should also be adequate to reach from the infusion site to a convenient location for the infusion device, without being so long as to hinder patient convenience with large amounts of loose tubing. For situations where the patient may be ambulatory during the infusion, a length in the range of 18 to 60 inches is typically appropriate, with a range of 24 to 36 inches being adequate for most applications.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

1. An adjustable infusion catheter for dispensing a medication or therapeutic fluid across a targeted region in a patient's body, the catheter comprising:
a. an elongated, flexible tube having an outside surface and outside diameter, said tube further including a proximal end and an opposite closed distal end; the tube contains one or more axial lumens that allows fluid to flow from the proximal end to the distal end of the tube, the distal end of the tube being arranged for insertion into the targeted region in the patient's body;
b. a source of low pressure, low flow medication or therapeutic fluid is attached to the proximal end of said flexible tube;
c. a fenestrated area is located near the distal end of the tube for dispensing said fluid from the lumen, across the outside surface of the tube and into the targeted region of the patient's body, said fenestrated area extending a predetermined length along the surface of the tube, the length of the fenestrated area being greater than the targeted region in the patient's body; and
d. an elongated flexible sheath having an inside diameter that is equal to or slightly greater than the outside diameter of the tube which allows the sheath to be slidably positioned along the outside surface of said tube to cover and close a predetermined length of the fenestrated area so as to leave an exposed length of the fenestrated area for dispensing the fluid over the corresponding targeted region in the patient's body.

2. An adjustable infusion catheter as defined in claim 1 wherein the sheath is formed from an opaque material so that the position of the sheath with respect to the fenestrated area can be visually determined.

3. An adjustable infusion catheter as defined in claim 1 where a bulb shaped portion is formed at the distal end of the tube so as to provide a stop to prevent the sheath from being moved beyond the end of said tube.

4. An adjustable infusion catheter as defined in claim 1 wherein indices are marked along a longitudinal axis of the tube whereby the proximal end of the sheath in association with the indices will identify the position of the sheath with respect to the fenestrated area to determine the exposed length of the fenestrated area.

5. An adjustable infusion catheter as defined in claim 1 wherein the tube and/or sheath has a low friction lubricious coating which allows the sheath to easily slide along the surface of the tube to allow it to be adjustably positioned with respect to the fenestrated area.

6. An adjustable infusion catheter as defined in claim 1 wherein the material used to form the tube and/or sheath has a low coefficient of friction which allows the sheath to easily slide along the surface of the tube to allow it to be adjustably positioned with respect to the fenestrated area.

7. An adjustable infusion catheter as defined in claim 1 wherein the sheath has a length which is greater than the length of the fenestrated area of said tube.

8. An adjustable infusion catheter as defined in claim 1 wherein each end of the sheath includes a fluid seal for sealing the ends of the sheath against said tube and still allow the sheath to be slidably positioned along the surface of the tube to adjust the exposed length of the fenestrated area.

9. An adjustable infusion catheter as defined in claim 8 wherein the sheath is formed from a heat shrinkable material and the circumferential end areas of the sheath are heated to reduce the inside diameter of the sheath to form the fluid seal against the outside surface of the tube whereby the fluid will not leak from said sheath.

10. An adjustable infusion catheter as defined in claim 8 wherein the fluid seal is a circumferential ring formed at each end of the sheath wherein the end rings have an inside diameter which is less than the outer diameter of the tube so as form a slidable fluid seal to prevent leakage between the sheath and the tube.

11. An adjustable infusion catheter as defined in claim 8 wherein an enlarged diameter circumferential collar is formed near the proximal end of the tube, the length of the sheath is equal to or less than the length of the tube from the collar to the distal end of the tube and the seal on the proximal end of the sheath slidably contacts the collar to prevent the distal end of the sheath from being moved beyond the distal end of said tube.

12. A method of making an adjustable infusion catheter for dispensing a medical or therapeutic fluid in a targeted area of a patient's body, the method including the following steps:
a. obtaining a predetermined length of elongated flexible tubing having an internal axial lumen and a proximal and distal end, said tubing having an outside diameter and an outside surface;
b. producing holes in an area of said tube near the distal end forming an elongated fenestrated area;
c. forming a tubular sheath having an inside diameter which is equal to or slightly greater than the outside diameter of said tube which allows the sheath to be slidably positioned over the outside surface of said tube;
d. slidably positioning said sheath over said tube;
e. forming at each end of the sheath a fluid tight seal between the sheath and the outside surface of the tube to prevent leakage of the fluid between the sheath and tube; and
f. slidably positioning the end of the sheath nearest the distal end of the tube over the fenestrated area of the tube to leave a predetermined exposed length which corresponds with the targeted area of the patient for evenly dispensing the fluid across the targeted area.

13. A method as described in claim 12 wherein the sheath is formed from a heat shrinkable material and a circumferential area at each end of the sheath is heated so that the ends of the sheath will shrink and fit tightly around the outside surface of the tube to form said fluid tight seal.

14. A method as described in claim 12 wherein the holes formed in the fenestrated area have slightly larger diameters as they get nearer to the distal end of the tube so that the fluid flow will be evenly dispensed along the predetermined exposed length of the fenestrated area.

15. An adjustable infusion catheter for evenly dispensing a medication or therapeutic fluid across a targeted region in a patient's body, the catheter comprising:
a. an elongated, flexible tube having an outside surface and outside diameter, said tube further including a proximal end and an opposite closed distal end;
b. the tube contains one or more axial lumens that allows fluid to flow from the proximal end to the distal end of the tube, the distal end of the tube being arranged for insertion into the targeted region in the patient's body;
C. the proximal end of said tube being connected to a source of low pressure, low flow medication or therapeutic fluid;
d. an elongated fenestrated area is formed near the distal end of the tube for dispensing said fluid from the lumen, across the outside surface of the tube and into the targeted region of the patient's body, said fenestrated area extending a predetermined length along the surface of the tube, the length of the fenestrated area being greater than the targeted region in the patient's body;
e. an elongated flexible sheath having an inside diameter that is equal to or slightly greater than the outside diameter of the tube which allows the sheath to be slidably positioned along the surface of said tube to cover and close a predetermined length of the fenestrated area so as to leave an exposed length for dispensing the fluid over the corresponding targeted region; and
f. the sheath is formed from a heat shrinkable material and the circumferential end areas of the sheath are heated to reduce the inside diameter of the sheath to form a fluid tight seal against the outside surface of the tube and still allow the sheath to be slidably adjusted with respect to said tube.
